(19) United States
(12) Patent Application Publication
(10) Pub. No.: US 2011/0144500 A1
(43) Pub. Date: Jun. 16, 2011

(54) ULTRASONIC DIAGNOSTIC APPARATUS AND METHOD FOR CALCULATING COORDINATES OF SCANNED SURFACE THEREOF

(75) Inventors: Koichi Nihei, Tokyo (JP); Dai Shinohara, Tokyo (JP)

(73) Assignee: HITACHI MEDICAL CORPORATION, Tokyo (JP)

(21) Appl. No.: 13/054,859

(22) PCT Filed: Jun. 25, 2009
(86) PCT No.: PCT/JP2009/061565

§ 371 Date: Jan. 19, 2011

(30) Foreign Application Priority Data
Jul. 22, 2008 (JP) 2008-189225

(51) Int. Cl. A61B 8/14

(52) U.S. Cl. 600/443

(57) ABSTRACT

An ultrasonic diagnostic apparatus includes a storage unit configured to store information about the contour of an object to be examined and information about the position of a specified region of the ultrasonic image of the object both corresponded to a body contour model coordinate system and a coordinate calculating unit configured to correspond the object coordinate system to the body contour model coordinate system according to the information about the position of the specified region of the object, associating with each other the object coordinate system and the body contour model coordinate system used when comparing an ultrasonic tomographic image based on ultrasonic tomographic data stored in an image storage unit with a real-time ultrasonic tomographic image scanned by an ultrasonic probe, and thereby calculating the coordinates of the scanned plane of the real-time ultrasonic tomographic image in association with the object coordinate system.

### FIELD OF THE INVENTION

The present invention relates to an ultrasonic diagnostic apparatus and method of calculating coordinates of the scanned surface thereof, in particular to a technique for making diagnosis by comparing two of the same cross-sectional images of an object that are imaged at different times.

### DESCRIPTION OF RELATED ART

An ultrasonic diagnostic apparatus is for making diagnosis of an imaged region noninvasively and in real time by transmitting/receiving ultrasonic waves to/from an object to be examined via a probe, reconstructing and displaying a tomographic image of the imaged region based on the reflected echo signals outputted from the probe.

Such ultrasonic diagnostic apparatus makes diagnosis by displaying and comparing, for example a tomographic image of a diseased area imaged before treatment (hereinafter referred to as a pre-treatment image) and a tomographic image of the diseased area imaged during or after the treatment (hereinafter referred to as a post-treatment image) to confirm the treatment effect of the diseased area. However, it generally is difficult to set the same conditions for the positional relationship of the object, etc. each time pre-treatment images and post-treatment images are scanned. Given this factor, it is necessary to estimate the imaging position of the pre-treatment image referring to the stored ultrasonic tomographic images, but comparing and displaying the same cross-sections in the tomographic images of an object in pre-treatment and post-treatment is difficult and time consuming.

Considering the above-mentioned point, for example Patent Document 1 discloses the method to acquire a pre-treatment image as the 3-dimensional volume data corresponding to the object coordinate system, extract and display the pre-treatment tomographic image in real time corresponding to the scanned plane of the post-treatment tomographic image from the acquired 3-dimensional volume data.

### PRIOR ART DOCUMENTS

Patent Document 1: JP-A-2005-296436

In the technique disclosed in Patent Document 1, the coordinate systems of an object before and after the treatment are corresponded to each other by setting a specified region (for example, an ensiform cartilage) as the original point and moving a probe to the position where the same cross-sections are scanned using the specified region as a guidance before scanning the post-treatment image.

However, since an ultrasonic tomographic image does not have body contour information of the object, it is difficult to estimate the position and the tilt of the probe at the time of scanning the tomographic image which makes it difficult to find the same cross-section thereof.

Given this factor, the present invention provides a technique to easily match the cross-sections of the ultrasonic tomographic images scanned before and after the treatment.

### BRIEF SUMMARY OF THE INVENTION

The ultrasonic diagnostic apparatus of the present invention comprises:

an ultrasonic probe configured to transmit/receive ultrasonic waves to/from an object to be examined;

a tomographic image data generating means configured to generate ultrasonic tomographic image data based on the reflected echo signals received by the ultrasonic probe;

a position detector configured to detect the position and tilt of the ultrasonic probe based on a sensor mounted to the ultrasonic probe;

a tomographic data storing unit configured to store the generated ultrasonic tomographic image data by corresponding the data to the object coordinate system set in advance in the object; and

a tomographic image data control unit, at the time that the ultrasonic tomographic image based on the stored ultrasonic tomographic image data and the real-time ultrasonic tomographic image scanned by an ultrasonic probe are compared, configured to calculate coordinates on the scanned plane of the real-time ultrasonic tomographic image in the object coordinate system set in advance in the object at the time of comparing the images based on the output of a position detector by corresponding to the object coordinate system.

Particularly, it has a body contour model provided with a setting that the contour information of the object and the positional information related to a specified region of the object are corresponded to the body contour model coordinate system, and is characterized that coordinates of the scanned plane of the real-time ultrasonic tomographic image are calculated in association with the object coordinate system, by corresponding the object coordinate system to the body contour model coordinate system based on the positional information related to the specified region of the object as well as corresponding the object coordinate system at the time of image comparison to the body contour model coordinate system.

Also, the ultrasonic diagnostic apparatus of the present invention is characterized in further comprising:

a storage unit configured to store the positional information related to contour information of the an object and a specified region on an ultrasonic image of the object by corresponding them to the body contour model coordinate system; and

a coordinate calculating unit configured to calculate coordinates of the scanned plane in a real-time ultrasonic tomographic image in association with the object coordinate system, by corresponding the object coordinate system to the body contour model coordinate system based on the positional information related to a specified region of the object as well as corresponding the object coordinate system at the time of comparing the ultrasonic tomographic image based on the ultrasonic tomographic image data stored in an image storage unit to the real-time ultrasonic tomographic image scanned by an ultrasonic probe with the body contour model coordinate system.

Also, the method for calculating coordinates of the scanned plane using the ultrasonic diagnostic apparatus related to the present invention is characterized in including:

a first step that stores the positional information related to contour information of an object and a specified region of an ultrasonic image of the object in a storage unit by corresponding them to the body contour model coordinate system; and

a second step that calculates, by a coordinate calculation unit, coordinates of the scanned plane of the real-time ultrasonic tomographic image in association with the object coordinate system, by corresponding the object coordinate system to the body contour model coordinate system based on the positional information related to a specified region of the object as well as associating the object coordinate system at the time that the ultrasonic tomographic image based on the ultrasonic tomographic image data stored in the image storage unit and the real-time ultrasonic tomographic image scanned by an ultrasonic probe are compared to the body contour model coordinate system.

That is, the body contour model in which the positional information related to the contour information of an object and the positional information related to a specified region of the object are set being corresponded to the body contour model coordinate system is prepared in advance, and stored in the storage unit. By associating the positional information of the specified region of the object at the time of storing data and the positional information of the specified region of the object at the time of comparing the images with the body contour model, it is possible to correspond the object coordinate system at the time of storing the data to the object coordinate system at the time of comparing the images.

In this manner, since coordinates of the scanned plane of the real-time ultrasonic tomographic image can be calculated in association with the object coordinate system at the time of storing the data even when the object coordinate system at the time of storing the data and the object coordinate system at the time of comparing the images are different, the display cross-section of the earlier-scanned tomographic image can be easily matched with the display cross-section of the later-scanned tomographic image.

Thus, in accordance with the present invention, it is possible to easily match the display cross-sections of the earlier-scanned tomographic image and the later-scanned tomographic image.

### BRIEF DESCRIPTION OF THE DIAGRAMS

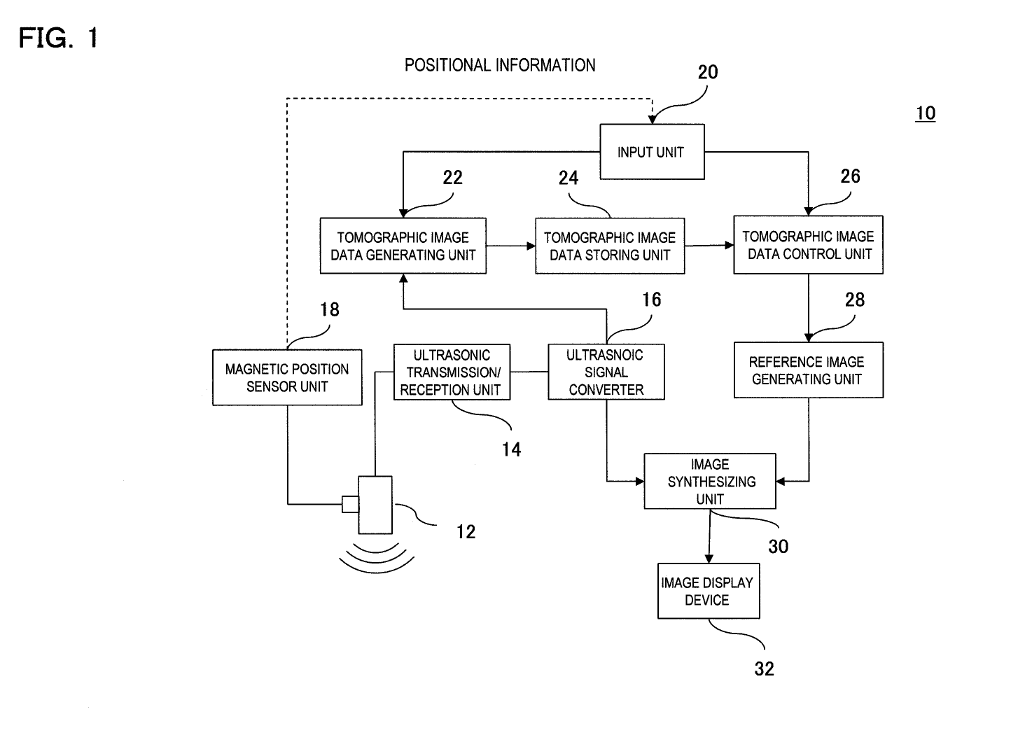

FIG. 1 shows a general block diagram of the ultrasonic diagnostic apparatus related to the present embodiment.

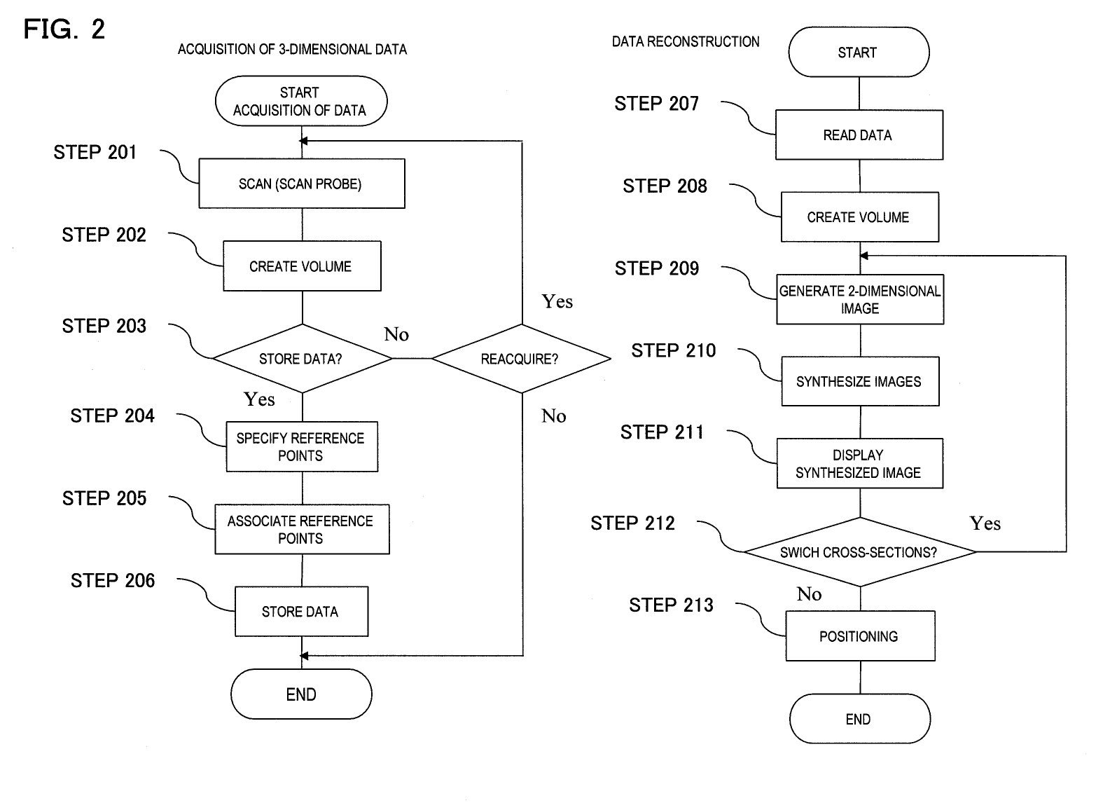

FIG. 2 is a flowchart of acquisition, storage and reconstruction process of 3-dimensional volume data in a first embodiment.

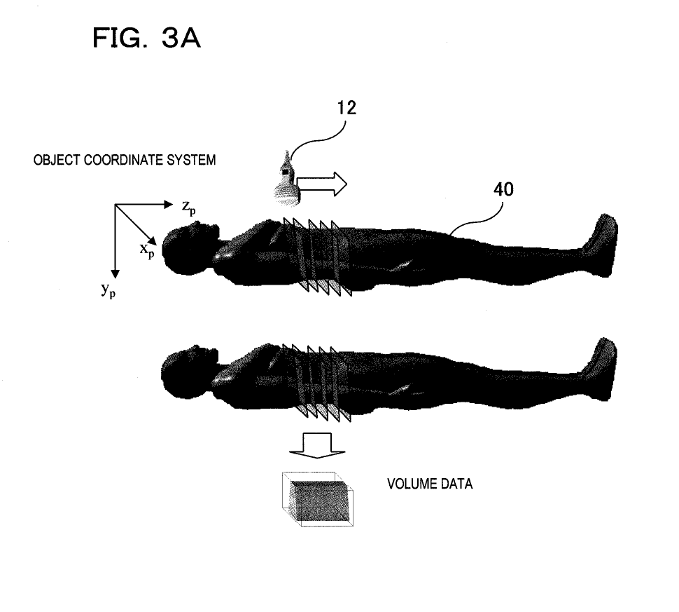

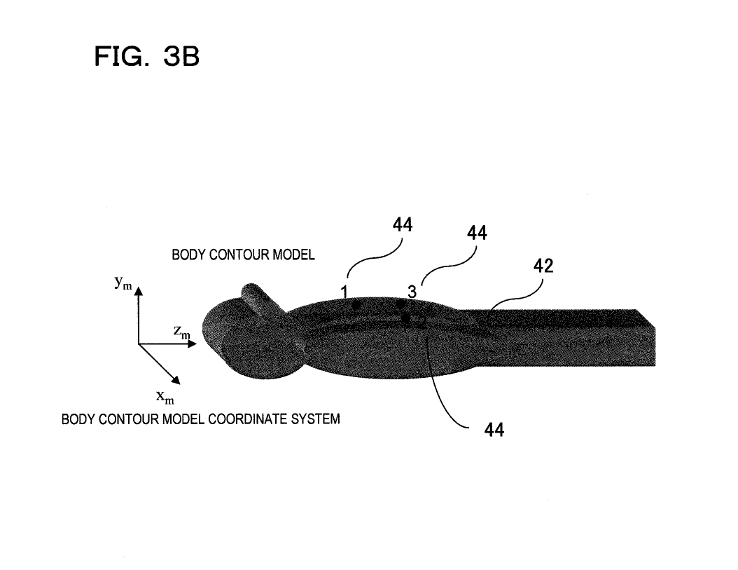

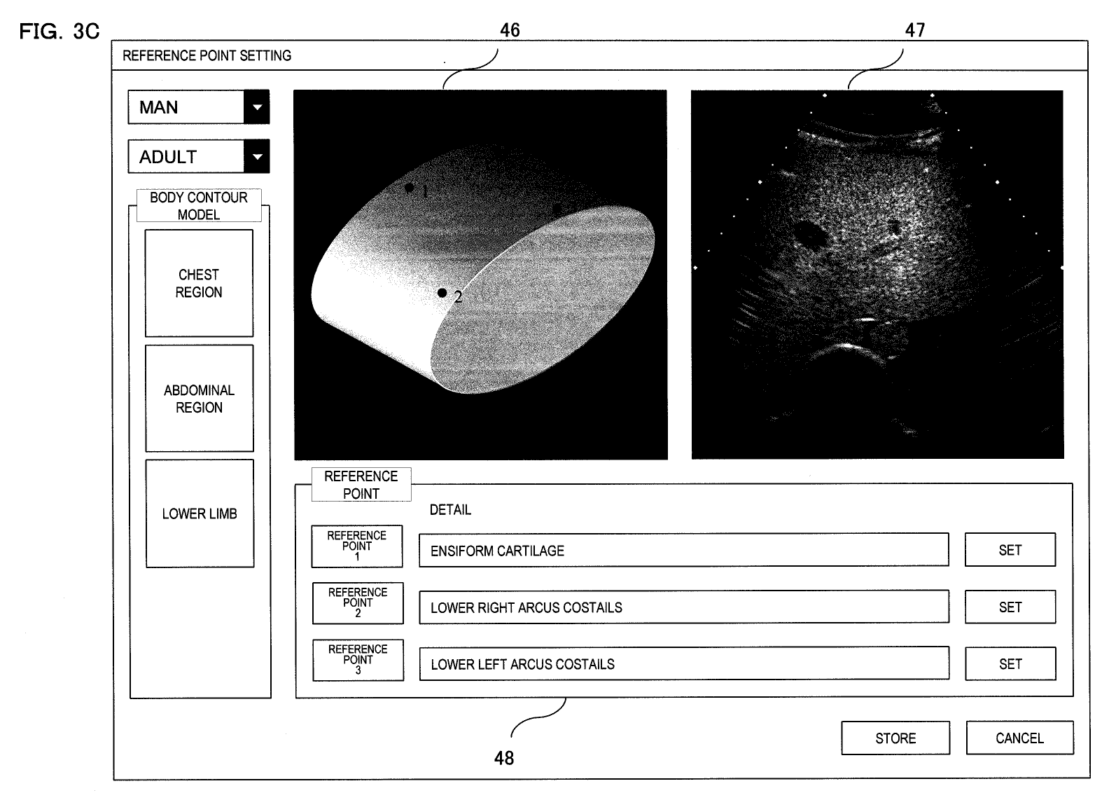

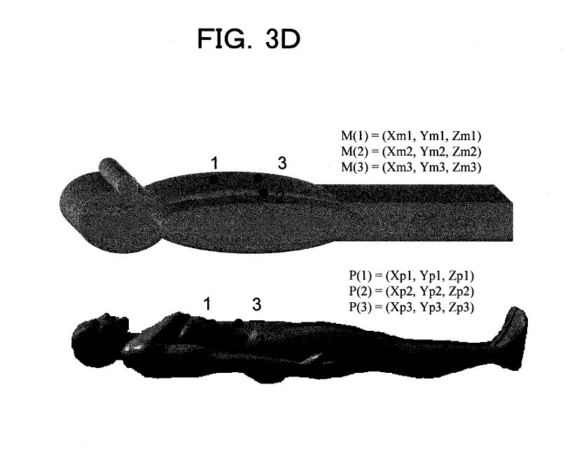

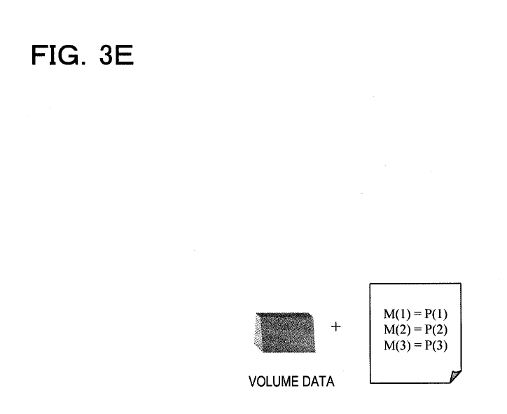

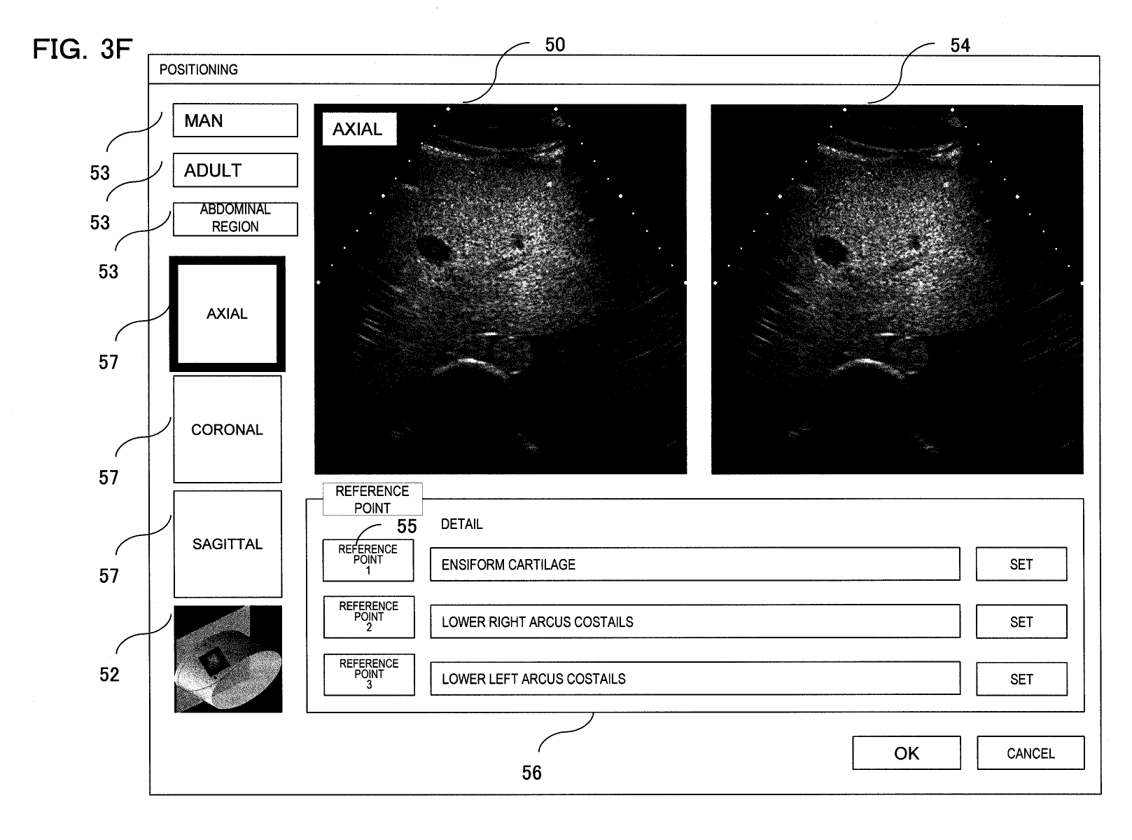

FIG. 3A is a schematic diagram for explaining acquisition of consecutive 2-dimensional ultrasonic tomographic images by scanning the body surface of an object, and generation of 3-dimensional volume data from the acquired plurality of 2-dimensional ultrasonic tomographic images.

FIG. 3B is a schematic diagram for explaining specification of three or more reference points correlated with a specified region of an object on a body contour model of the object.

FIG. 3C is a display example of an image display device for setting the reference points.

FIG. 3D is a schematic diagram for explaining the association between the 3-dimensional coordinates of the respective reference points in body contour model coordinate system M and the 3-dimensional coordinates of the corresponding region in object coordinate system P.

FIG. 3E is a schematic diagram for explaining storage of the associated positional information and the volume data.

FIG. 3F is a display example on an image display device at the time of executing positioning of coordinates of the respective reference points in body contour model coordinate system M and the coordinates to be correlated with the respective reference points in object coordinate system P′ at the time of comparing images (at the time of scanning a post-treatment tomographic image).

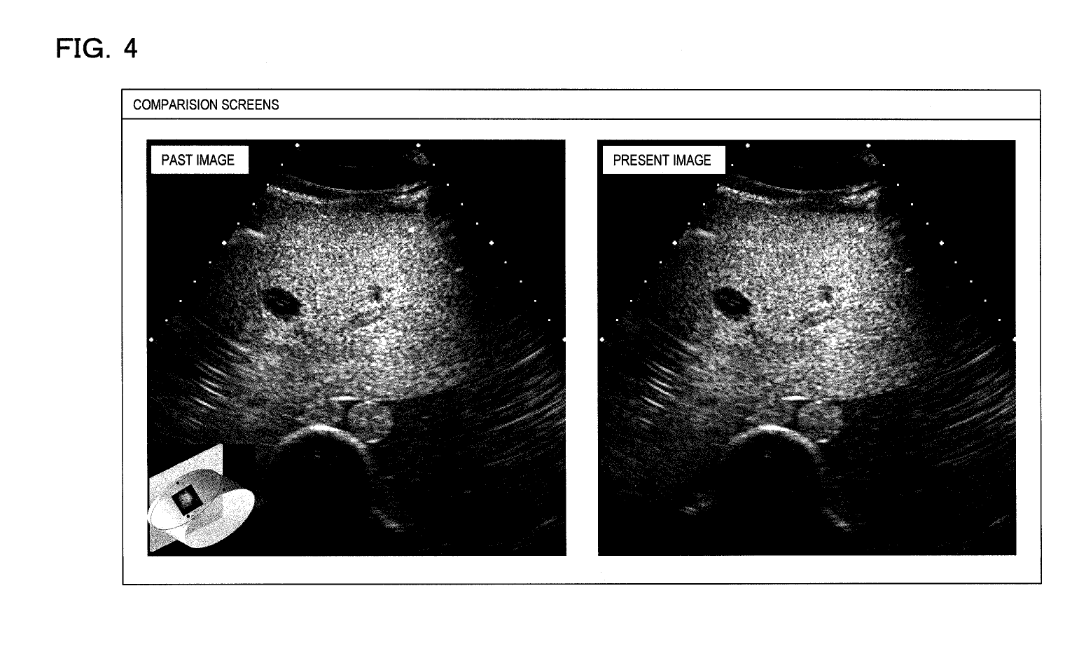

FIG. 4 shows a display example of an image display device at the time of comparing the pre-treatment tomographic image and the post-treatment tomographic image.

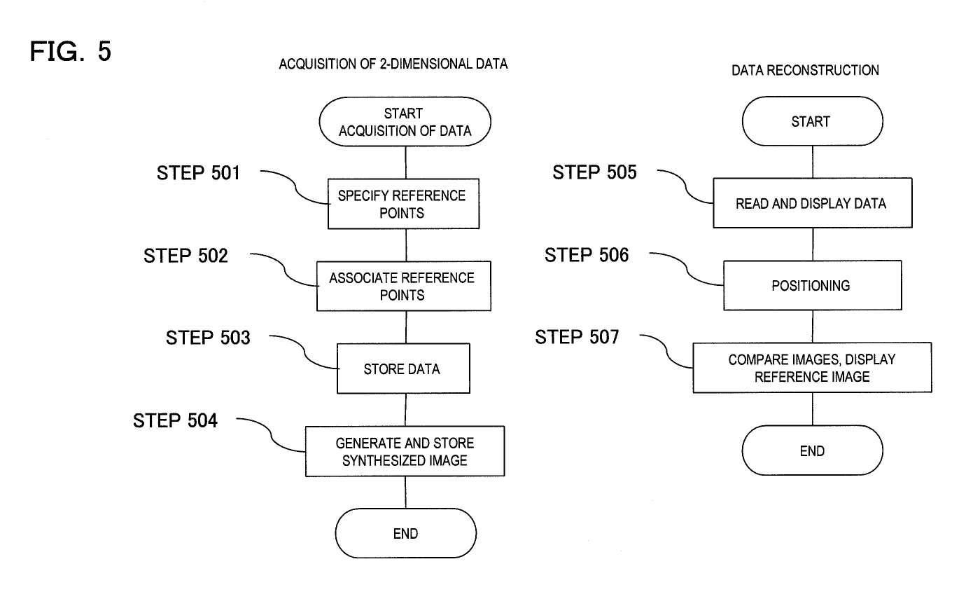

FIG. 5 is a flowchart showing acquisition, storage and reconstruction process of the 2-dimensional ultrasonic tomographic image data in a second embodiment.

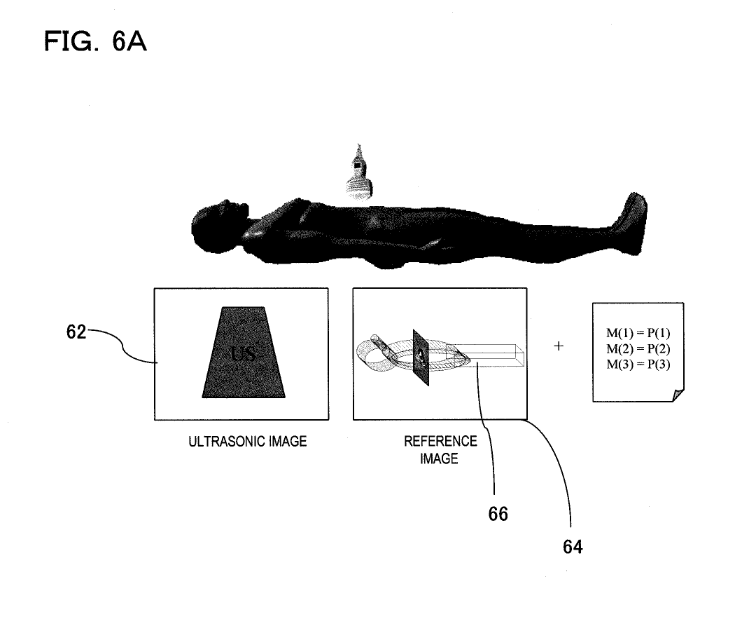

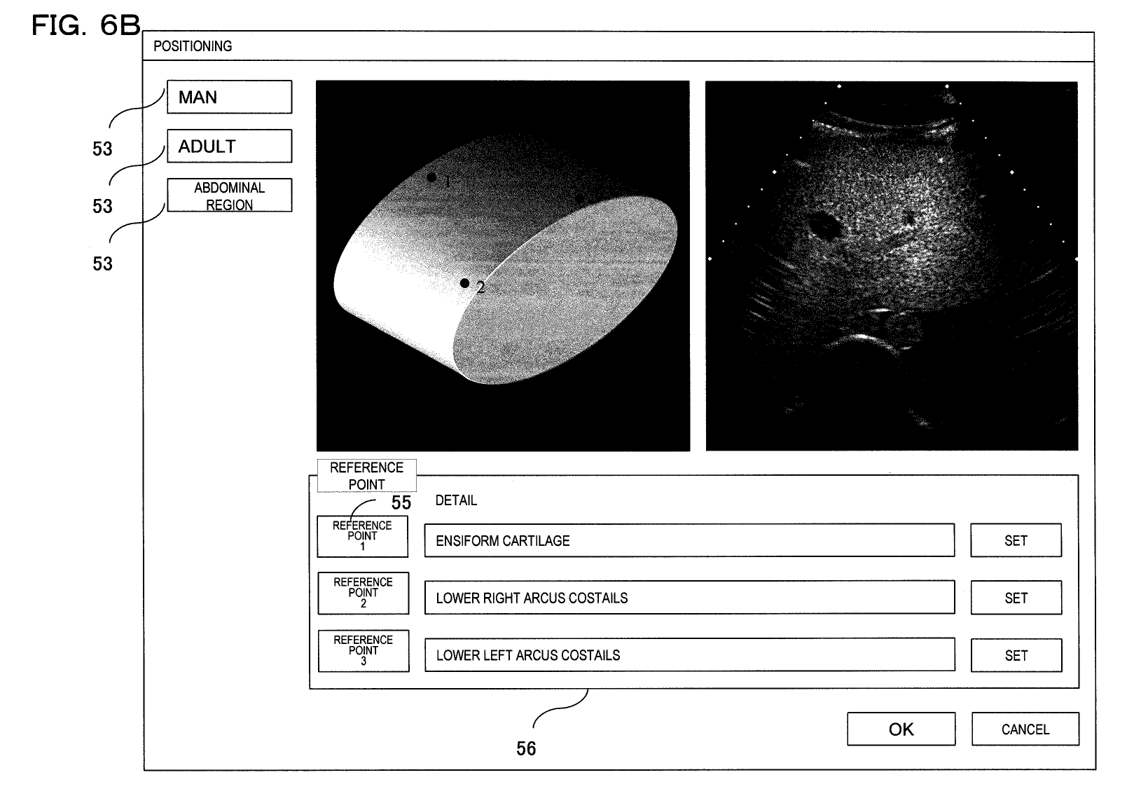

FIG. 6A is a schematic diagram explaining storage of an ultrasonic tomographic image, correlated positional information and a reference image.

FIG. 6B is a display example on an image display device at the time of executing positioning of coordinates of the respective reference points in body contour model coordinate system M and the coordinates to be correlated with the respective reference points in object coordinate system P′ at the time of comparing the images (at the time of scanning a post-treatment tomographic image).

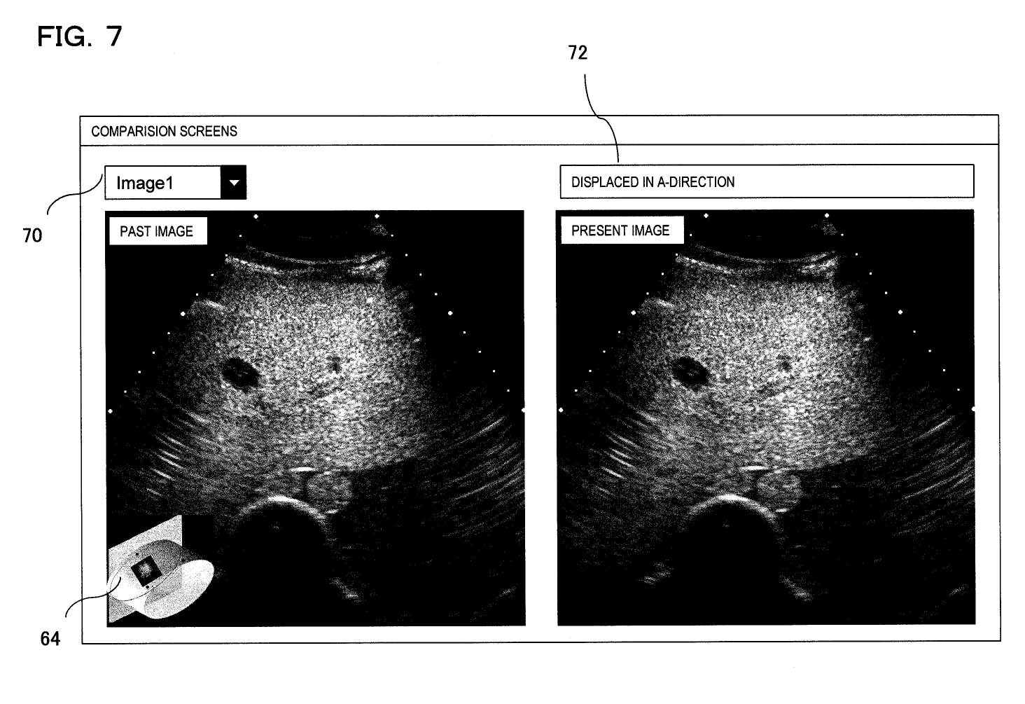

FIG. 7 is a display example of image display device 32 at the time of comparing a pre-treatment image and a post-treatment image.

### DETAILED DESCRIPTION OF THE INVENTION

An embodiment of the ultrasonic diagnostic apparatus to which the present invention is applied will be described below. In the following description, the same function parts are represented by the same reference numerals, and the duplicative description thereof is omitted.

FIG. 1 is a general block diagram of the ultrasonic diagnostic apparatus related to the present embodiment. Ultrasonic diagnostic apparatus 10 of the present embodiment comprises:

ultrasonic probe 12 configured to transmit/receive ultrasonic waves to/from an object to be examined;

ultrasonic transmission/reception unit 14 configured to transmit driving signals to ultrasonic probe 12 and processes the reflected echo signals received by ultrasonic probe 12; and

ultrasonic signal converter 16 configured to execute luminance conversion, etc. on the ultrasonic signals acquired from ultrasonic transmission/reception unit 14.

Also, as the position detector for detecting the position and tilt of ultrasonic probe 12, a source origin such as a transmitter and magnetic position sensor unit 18 that includes a device such as a magnetic positional sensor mounted to ultrasonic probe 12 for detecting the magnetic field generated from the source origin are provided. Also, input unit 20 to which the positional information from magnetic position sensor unit 18 is inputted is provided. As for the position detector, various commonly-known techniques capable of detecting the position and tilt of ultrasonic probe 12 can be used without being limited to the above-mentioned technique.

Also, the ultrasonic diagnostic apparatus of the present embodiment further comprises:

tomographic image data generating unit 22 configured to generate 2-dimensional ultrasonic tomographic data based on the output from ultrasonic signal converter 16 and the positional information of ultrasonic probe 12 inputted to input unit 20 or 3-dimensional volume data formed by plural sets of 2-dimensional ultrasonic tomographic data; and

tomographic data storing unit 24 configured to store the ultrasonic tomographic data generated in tomographic data generating unit 22 being associated with a pre-set body contour model of the object in storage means. The body contour model is a pre-set data of the contour information of the object and the positional information related to a specified region being corresponded to the body contour model coordinate system.

Also, it comprises tomographic image data control unit configured to associate the coordinate system of the ultrasonic tomographic data at the time of storing data with the coordinate system of the ultrasonic tomographic image at the time of comparing the images based on the positional information of ultrasonic probe 12 inputted to input unit 20 and the positional information of the body contour model.

Also, it comprises:

reference image generating unit 28 configured to construct a 2-dimensional ultrasonic tomographic image based on the information obtained by tomographic image data control unit 26 (hereinafter referred to as a virtual tomographic image or reference tomographic image);

image synthesizing unit 30 configured to construct a real time 2-dimensional image (ultrasonic tomographic image) based on the signals generated by ultrasonic signal converter 16, and synthesizes the real-time 2-dimensional image with the 2-dimensional tomographic image (reference tomographic image) constructed by reference image generating unit 28; and

image display device 32 such as a monitor that displays the image synthesized by image synthesizing unit 30.

Such configured ultrasonic diagnostic apparatus of the present embodiment is for comparing and displaying the pre-treatment image and the post-treatment image for making diagnosis, for example to confirm treatment effect of the object's diseased area. Generally, since the object receives ultrasonic examination again several days after the scanning of a pre-treatment image, it is not easy to search for the same cross-section of the post-treatment tomographic image while observing the pre-treatment tomographic image. The ultrasonic diagnostic apparatus of the present embodiment provides a technique to easily match the displayed cross-sections of the earlier-scanned tomographic image and the later-scanned tomographic image. Characteristic parts of the ultrasonic diagnostic apparatus related to the present invention will be described below by different examples.

### Embodiment 1

The present embodiment stores the 3-dimensional volume data formed by plural sets of ultrasonic tomographic image data in a storage means, extracts the ultrasonic tomographic image of the same cross-section as the present ultrasonic scanned plane from the stored 3-dimensional volume data, and displays the extracted image along with the real-time ultrasonic tomographic image.

FIG. 2 is a flowchart showing acquisition, storage and reconstruction process of the 3-dimensional volume data in the present embodiment. FIG. 3 shows a schematic diagram and display example, etc. in each process of the present embodiment.

As shown in FIG. 3A, the body surface of object 40 is scanned by ultrasonic probe 12 to which a magnetic position sensor is attached, consecutive 2-dimensional ultrasonic tomographic images are obtained (step 201), and the 3-dimensional volume data is generated from a plurality of 2-dimensional tomographic images (step 202). In the case of storing the 3-dimensional volume data, the following data storage process is started (step 203).

First, in the data storage process, body contour model 42 of the object which is created in advance and stored in a device such as storage means is displayed on image display device 32, and a user such as a doctor or examination operator specifies (sets) at least three reference points 44 which are correlated with a specified region of the object on the body contour model (step 204) as shown in FIG. 3B.

In concrete terms, as shown in a display example of image display device 32 of FIG. 30 at the time of setting the reference points, the body contour model can be prepared for various types of patients such as adult/child or male/female, and these types can be selected from a pull-down menu. Also, it can be prepared for each region of the object such as a neck region, heart or lower limb. When these types are selected, the selected body contour model 42 is displayed on display section 46. Also, the body contour model can be prepared for each type of ultrasonic probes. In display section 46, an operation such as rotation of the body contour model can be executed.

A user sets reference points on body contour model 42. For example, the reference points can be set by repeating the operation three times to push the button of “reference point 1” in reference point setting menu 48, specify reference points 44 manually on body contour model 42 and input detailed information of reference point 44 in a detailed information input column. Also, for example specified regions of an object in which the reference points can be set using a pull-down menu may be prepared in advance so that the reference points can be automatically set on body contour model 42 and the detailed information thereof is inputted, by corresponding to the specified region specified by a user referring to the pull-down menu.

After reference points 44 are set as described above, the body contour model and the object are associated with each other using the positional information of the three points specified in step 204 (step 205). In concrete terms, the real-time ultrasonic tomographic image is displayed on display section 47 as a reference for associating the body contour model with the object, and the coordinates correlating with the specified region of the object (for example, the coordinate of the ultrasonic probe) can be associated with the coordinates correlating with the specified region in the body contour model (for example, the coordinates on the body contour) by moving the ultrasonic probe to the position where the specified region of the object (for example, ensiform cartilage, etc.) is scanned and pushing a setting button in reference setting menu 48.

By repeating this process for the respective reference points, three-dimensional coordinates M(1):(Xm1, Ym1, Zm1), M(2):(Xm2, Ym2, Zm2), M(3):(Xm3, Ym3, Zm3) of the respective reference points in body contour model coordinate system M can be corresponded to and associated with 3-dimensional coordinates P(1):(Xp1, Yp1, Zp1), P(2):(Xp2, Yp2, Zp2), P(3):(Xp3, Yp3, Zp3) of the corresponding region in object coordinate system P respectively as shown in FIG. 3D.

In this manner, by executing the correspondence of coordinates in at least three points, body contour model coordinate system M can be corresponded to object coordinate system P at the time of storing data, whereby making it possible to identify the correlation between them.

Next, as shown in FIG. 3E, the associated positional information (association of the coordinates showing the relationship between body contour model coordinate system M and object coordinate system P) and the volume data are stored (step 206). For example, the storage can be executed by pushing a storage button in reference point setting menu 48.

In this manner, generation and storage of the 3-dimensional volume data is completed. Then ultrasonic scanning of an object is executed again, for example to confirm the treatment effect of the object's diseased area at a later date. The operation for displaying the same cross-section at the later date will be described below.

First, the stored positional information and 3-dimensional volume data are read in (step 207), and the 3-dimensional volume data is reconstructed (step 208). Then a 2-dimensional image of an given cross-section (reference tomographic image) is constructed from the 3-dimensional volume data (step 209), and a reference image in which reference points 44, the ultrasonic scanned plane passing through the reference points and the reference tomographic image in the scanned plane are synthesized is created from body contour model 42, positional information and the reference tomographic image (step 210).

Next, the reference tomographic image and the reference image are displayed on image display device 32 (step 211). In concrete terms, for example the reference tomographic image is displayed on display section 50 and the reference image is displayed on display section 52 as shown in FIG. 3F. FIG. 3F is a display example of image display device 32 at the time of positioning coordinates of the respective reference points in body contour model coordinate system M and the coordinates correlated with the respective reference points in object coordinate system P′ at the time of comparing the images (at the time of scanning the post-treatment tomographic image).

The cross-sections of the reference tomographic image and the reference image are arbitrarily switched (step 212), and steps 209˜211 are repeated every time the cross-sections are switched. In concrete terms, image display device 32 is provided with button 55 for selecting the respective reference points in positioning menu 56 and the respective buttons 57 for selecting a direction from among axial, coronal and sagittal are provided, and is capable of selecting the cross-section in an arbitrary direction of an arbitrary reference point and switching the cross-sections of the reference tomographic image and the reference image to be displayed. On display section 53, condition of the stored data is displayed.

Next, the user executes positioning of the ultrasonic tomographic image scanned in real time and the reference tomographic image, referring to the reference image and the reference tomographic image (step 213). In concrete terms, he/she performs scanning while changing the position of ultrasonic probe 12 so that the ultrasonic tomographic image displayed on display section 54 in real time becomes the same image as the reference tomographic image displayed on display section 50.

Here, in the present embodiment, the body contour model having the body contour information of the object is displayed for reference, and the ultrasonic scanned plane in which the reference tomographic image is reconstructed on the body contour model is also displayed. Therefore, the user can visually identify which cross-section of the object is displayed as the reference tomographic image on display section 50, whereby making it easy for him/her to search for the real-time ultrasonic tomographic image which is the same as the reference tomographic image.

After obtaining the same ultrasonic tomographic image, the operation to push the setting button in positioning menu 56 is executed for each reference point. In this manner, coordinates of the respective reference points in body contour model coordinate system M is associated with the coordinates correlated with the respective points in object coordinate system P′ at the time of comparing the images (at the time of scanning the post-treatment image), which makes it easier for the user to identify the relationship between body contour model coordinate system M and object coordinate system P′ at the present time. When positioning of the three reference points is completed, the process moves on to the mode for comparing the pre-treatment tomographic image and the post-treatment tomographic image by pushing an OK button on image display device 32.

FIG. 4 is a display example of image display device 32 at the time of comparing the pre-treatment tomographic image and the post-treatment tomographic image. The left side displays the past image (the reference tomographic image clipped and constructed from the 3-dimensional volume data) and the right side displays the present image (the ultrasonic tomographic image in real time). Also, as shown in FIG. 4, the reference image in which the body contour model, the respective reference points, the ultrasonic scanned plane and the reference tomographic image in the scanned plane are synthesized may be displayed along with the past image.

By the above-described positioning operation, the relationship between object coordinate system P at the time of scanning and storing the 3-dimensional volume data and object coordinate system P′ at the time of comparing the images (at the time of scanning the post-treatment tomographic image) can be identified by corresponding to each other via body contour model coordinate system M. Therefore, the coordinates on the same cross-section as the ultrasonic scanned plane of the real-time ultrasonic tomographic image is calculated in association with object coordinate system P, and the ultrasonic tomographic image of the same cross-section as the calculated coordinates is extracted and constructed from the 3-dimensional volume data.

As a result, user can consistently refer to the past image of the same cross-section as the real-time ultrasonic tomographic image while executing scanning with respect to the object using the ultrasonic probe, whereby making it possible to easily make diagnosis of the object's condition before and after the treatment. In this manner, in accordance with the present embodiment, it is possible to easily match the displayed cross-sections of the earlier-scanned ultrasonic tomographic image and the one scanned afterward.

### Embodiment 2

In the present embodiment, not 3-dimensional volume data formed by plural sets of ultrasonic tomographic data but one set or plural sets of ultrasonic tomographic data which do not construct 3-dimensional volume data are stored in storage means, and position of an ultrasonic probe is guided for displaying the real-time ultrasonic tomographic image of the same cross-section as the stored ultrasonic tomographic image data. The difference from the first embodiment will be mainly described below and the duplicative description thereof will be appropriately omitted.

FIG. 5 is a flowchart of acquisition, storage and reconstruction process of 2-dimensional ultrasonic tomographic image data in the present embodiment. FIG. 6 shows schematic diagrams, display example, etc. in each process of the present embodiment.

First, discriminative three points are specified on body contour model 42 by the same operation in the first embodiment (step 501), and the body contour model and the object are associated with each other by the positional information of the three points specified in step 501 (step 502).

Next, as shown in FIG. 6A, arbitrary ultrasonic tomographic data is selected from among the scanned ultrasonic tomographic data, and the selected ultrasonic tomographic data 62, positional information used for association (association of the coordinates showing the relationship between body contour model coordinate system M and object coordinate system P at the time of storing the data) and reference image 64 are stored (steps 503 and 504). Here, reference image 64 is constructed by body contour model 42, the scanned plane of ultrasonic tomographic data 62, the ultrasonic tomographic image data in the scanned plane and so on.

In this way, generation and storage of the 2-dimensional ultrasonic tomographic image is completed. And for example, at a later date, ultrasonic scanning is executed again on the object to confirm the treatment effect in the diseased area. The operation for displaying the same cross-sections at that time will be described below.

First, the stored ultrasonic tomographic data, the reference image, etc. are read in, and the ultrasonic tomographic image, the body contour model and the respective reference points on the model are displayed as shown in FIG. 6B (step 505). Then the stored reference points and the positional information are associated with each other while the user is referring to the ultrasonic tomographic image (step 506).

In this manner, coordinates of the respective reference points in body contour model coordinate system M is associated with the coordinates correlating with the respective reference points in object coordinate system P′ at the time of comparing the images (at the time of scanning the post-treatment image), and the relationship between body contour model coordinate system M and object coordinate system P′ at the time of comparing the images is identified. Next, the process proceeds to the mode for comparing the pre-treatment image and the post-treatment image.

FIG. 7 is a display example of image display device 32 at the time of comparing the pre-treatment image and the post-treatment image. The left-side of the display is the past image, and the right-side of the display is the present image (real-time ultrasonic tomographic image). Also, as shown in FIG. 4, the reference image in which the body contour model, the respective reference points, the ultrasonic scanned plane and the reference tomographic image in the scanned plane are synthesized may be displayed along with the past image.

As shown in FIG. 7, the reference image is displayed along with the past image, and the past image (pre-treatment image) and the present image (post-treatment image) are compared (step 507). In concrete terms, in the case that there are plural sets of stored ultrasonic tomographic data, the ultrasonic tomgoraphic data to be compared is selected from a pull-down menu 70, and the ultrasonic tomographic image based on the selected ultrasonic tomographic data is displayed.

Guidance display 72 is displayed for the ultrasonic probe to be moved to the position where the real-time ultrasonic tomographic image of the same cross-section as the tomographic data can be scanned. Concretely, a message such as “displaced in XX-direction” or “move in XX-direction” is displayed.

The user moves ultrasonic probe 12 according to the guidance display. Then when ultrasonic probe 12 is positioned at the same cross-section as the stored ultrasonic tomographic data, the completion of positioning is indicated by the guidance display, then the comparison of the pre-treatment image and the post-treatment image is executed in this condition.

In this manner, in accordance with the present embodiment, since the body contour model having the body contour information of the object is displayed as reference, the user can easily execute positioning of body contour model coordinate system M and object coordinate system P′ at the time of comparing the images. As a result, the relationship between object coordinate system P at the time of storing data and object coordinate P′ at the time of comparing the images can be corresponded to each other and indicated via body contour model coordinate system M, and the position of the ultrasonic probe for obtaining the real-time ultrasonic tomographic image of the same cross-section as the pre-treatment ultrasonic tomographic image can be guided. Therefore, the cross-sections to be displayed of the earlier-scanned tomographic image and the later-scanned tomographic image can be easily matched.

While the case that ultrasonic scanning is executed in the condition that the object is laying on his/her back is exemplified in the first and second embodiments, the position of the object is not limited thereto. As long as the posture of the object is the same at the time that the data is stored and the time that the effect of treatment is confirmed later on, the relationship between object coordinate systems P and P′ can be corresponded and grasped as in the first and second embodiments even when the object is in a given posture such as laying on his/her side or stomach.

Also, the tomographic data storing unit may include means to set at least three reference points related to a specified region of an object on the contour of a body contour model displayed on an image display and means to associate coordinates in the body contour model coordinate system of the respective reference points set on the body contour model with coordinates in the object coordinate system of the region corresponding to the respective reference points of the object and store the associated coordinates, and the tomographic data control unit may include means to associate coordinates in the body contour model coordinate system in the respective reference points set on the body contour model with coordinates in the object coordinate system of the region corresponding to the respective reference points of the object at the time of comparing the images.

In this manner, by executing the association of at least three points of the coordinates related to a specified region of the object, it is possible to correspond the object coordinate system at the time of storing the data to the body contour model coordinate system as well as corresponding the object coordinate system at the time of comparing the images to the body contour model coordinate system.

Also, the ultrasonic diagnostic apparatus may further comprise, in the case that the stored ultrasonic tomographic data are the 3-dimensional volume data formed by plural sets of ultrasonic tomographic image data:

a reference image generating unit configured to extract the tomographic image data calculated by the tomographic image data control unit or corresponding to coordinates of the scanned plane of the real-time ultrasonic tomographic image from the 3-dimensional volume data and generate a reference tomographic image;

an image synthesizing unit configured to synthesize the real-time ultrasonic tomographic image and the reference tomographic image; and

an image display configured to display the synthesized real-time ultrasonic tomographic image and the reference tomographic image.

In this manner, the user can consistently refer to the reference tomographic image (past image) of the same cross-section as the real-time ultrasonic tomographic image while executing scanning with respect to the object using the ultrasonic probe, whereby making it possible to easily make diagnosis of, for example the condition of the diseased area of the object in the pre-treatment and post-treatment.

Also, the ultrasonic diagnostic apparatus may further comprise means to calculate moving direction of the ultrasonic probe for superimposing the scanned plane of the calculated real-time ultrasonic tomographic image to the cross-sectional plane of the stored ultrasonic tomographic image data based on coordinates of the cross-sectional plane of the stored ultrasonic tomographic image data and coordinates of the scanned plane of the real-time ultrasonic tomographic image calculated by the tomographic image data control unit, and to display the calculated moving direction on an image display.

In this manner, in the case that the stored ultrasonic tomographic image data is not the 3-dimensional volume data but 2-dimensional ultrasonic tomographic image data for one or plural images, it is possible to provide positional guidance to which direction the ultrasonic probe should be moved for obtaining the real-time ultrasonic tomographic image of the same cross-section as the ultrasonic tomographic image based on the stored ultrasonic tomographic image data. Therefore, the cross-sections to be displayed of the earlier-scanned tomographic image and the later-scanned tomographic image can be easily matched.

The preferable embodiments of the ultrasonic diagnostic apparatus according to the present invention have been described referring to the attached drawings. However, the present invention is not limited to these embodiments. It is obvious that persons skilled in the art can make various kinds of alterations or modifications within the scope of the technical idea disclosed in this application, and it is understandable that they belong to the technical scope of the present invention.

### DESCRIPTION ON THE REFERENCE NUMERALS

10: ultrasonic diagnostic apparatus, 12: ultrasonic probe, 18: magnetic position sensor unit, 22: tomographic image data generating unit, 24: tomographic image data storing unit, 26: tomographic image data control unit, 28: reference image generating unit, 30: image synthesizing unit, 32: image display device, 42: body contour model, 44: reference points, 62: ultrasonic tomographic data, 64: reference image, 72: guidance display

1. An ultrasonic diagnostic apparatus comprising:
a storing unit configured to store positional information related to contour information of an object to be examined and a specified region of an ultrasonic image of the object by corresponding them to the body contour model coordinate system; and
a coordinate calculating unit configured to calculate coordinates of the scanned plane of a real-time ultrasonic tomographic image in association with the object coordinate system, by corresponding the object coordinate system to the body contour model coordinate system based on the positional information related to the specified region of the object as well as corresponding the object coordinate system at the time of comparing the ultrasonic tomographic image based on the ultrasonic tomographic image data stored in an image storage unit with the real-time ultrasonic tomographic image scanned by an ultrasonic probe to the body contour model coordinate system.

2. An ultrasonic diagnostic apparatus comprising:
an ultrasonic probe configured to transmit/receive ultrasonic waves to/from an object to be examined;
a tomographic image data generating unit configured to generate ultrasonic tomographic image data based on the reflected echo signals received by the ultrasonic probe;
a position detecting unit configured to detect position and tilt of an ultrasonic probe based on a sensor mounted to the ultrasonic probe;
a tomographic image data storing unit configured to store the generated ultrasonic tomographic image data based on the output from the position detecting unit by corresponding the data to the object coordinate system set in advance in the object; and
a tomographic image data control unit, at the time of comparing the ultrasonic tomographic image based on the stored ultrasonic tomographic image data with the real-time ultrasonic tomographic image scanned by the ultrasonic probe, configured to calculate coordinates of the scanned plane of the real-time ultrasonic tomographic image in the object coordinate system which is set in advance in the object at the time of comparing the images in association with the object coordinate system based on the output from the position detecting unit,
characterized in further comprising a coordinate calculating unit having a body contour model in which the positional information related to the contour information of the object and a specified region of the object are corresponded to the body contour model coordinate system, configured to calculate coordinates of the scanned plane of the real-time ultrasonic tomographic image in association with the object coordinate system, by corresponding the object coordinate system to the body contour model coordinate system based on the positional information related to the specified region of the object as well as corresponding the object coordinate system at the time of comparing the images to the body contour model coordinate system.

3. The ultrasonic diagnostic apparatus according to claim 2, characterized in comprising a reference point setting unit configured to set at least three reference points related to a specified region of the object on a contour of the body contour model displayed on an image display, wherein:
the tomographic data storing unit associates coordinates in the body contour model coordinate system of the respective reference points set on a body contour model with coordinates in the object coordinate system of the region corresponding to the respective reference points of the object and stores the associated coordinates; and
the coordinate calculating unit calculates coordinates of the scanned plane of the real-time ultrasonic tomographic image by associating coordinates in the body contour model coordinate system of the respective reference points set on the body contour model with coordinates in the object coordinate system of the region corresponding to the respective reference points of the object at the time of comparing the images.

4. The ultrasonic diagnostic apparatus according to claim 2, wherein the stored ultrasonic tomographic data is the 3-dimensional volume data formed by plural sets of ultrasonic diagnostic image data, characterized in further comprising:
a reference image generating unit configured to extract the tomographic image data corresponding to the coordinates of the scanned plane of the real-time ultrasonic tomographic image calculated by the tomographic image data control unit from the 3-dimensional volume data and generate a reference tomographic image;
an image synthesizing unit configured to synthesize the real-time ultrasonic tomographic image and the reference tomographic image; and
an image display configured to display the synthesized real-time ultrasonic tomographic image and the reference tomographic image.

5. The ultrasonic diagnostic apparatus according to claim 2, characterized in further comprising a display control unit configured to calculate moving direction of the ultrasonic probe for superimposing the scanned plane of the calculated real-time ultrasonic tomographic image to the cross-sectional plane of the stored ultrasonic tomographic image data based on the coordinates of the cross-sectional plane of the stored ultrasonic tomographic image data and the coordinates of the scanned plane of the real-time ultrasonic tomographic image data calculated by the tomographic image data control unit, and control the display of the calculated moving direction on the image display.

6. The ultrasonic diagnostic apparatus according to claim 2, wherein the body contour model, the respective reference points set on the contour of the body contour model thereof, a scanned plane of the ultrasonic tomographic image passing through the reference points of the body contour model and an ultrasonic tomographic image on the scanned plane are displayed.

7. A method for calculating coordinates of a scanned plane of the ultrasonic diagnostic apparatus including:
a first step that stores contour information of an object to be examined and positional information related to a specified region of an ultrasonic image of the object in a storage unit; and
a second step that calculates, by a coordinate calculating unit, coordinates of a scanned plane of the real-time ultrasonic tomographic image in association with the object coordinate system, by corresponding the object coordinate system to the body contour model coordinate system based on the positional information related to a specified region of the object as well as corresponding the object coordinate system at the time of comparing the ultrasonic tomographic image based on the ultrasonic tomographic image data stored in an image storage unit with the real-time ultrasonic tomographic image scanned by an ultrasonic probe to the body contour model coordinate system.
